(19) United States
(12) Patent Application Publication
(10) Pub. No.: US 2022/0080422 A1
(43) Pub. Date: Mar. 17, 2022

(54) MICROFLUIDIC PARTICLE CONCENTRATORS

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 17/417,615

(22) PCT Filed: Feb. 12, 2019
(86) PCT No.: PCT/US2019/017633

§ 371 Date: Jun. 23, 2021

(51) Int. Cl. B01L 3/00

(52) U.S. Cl. CPC B01L 3/502761; B01L 2400/0478; B01L 2300/0681

(57) ABSTRACT

The present disclosure relates to a microfluidic particle concentrator that includes an inlet microchannel, a filtering chamber fluidly connected to the inlet microchannel to receive a sample fluid, and a mechanical filter positioned in the filtering chamber. The particle concentrator also includes a filter outlet microchannel fluidly connected to the filtering chamber to receive a particle-ablated fluid formed by passing through the mechanical filter, a particle outlet microchannel fluidly connected to the filtering chamber to receive a particle-concentrated fluid including a plurality of particles not permitted to pass through the mechanical filter, and a fluid movement network including multiple pumps. The multiple fluid pumps generate sample fluid flow through the inlet microchannel and into the filtering chamber, particle-ablated fluid flow from the mechanical filter into the filter outlet microchannel, and particle-concentrated fluid from the filtering chamber into the particle outlet microchannel.

### BACKGROUND

In biomedical, chemical, and environmental testing, the ability to separate and/or concentrate undissolved particles from liquids can be desirable. As the quantity of available assays for undissolved particles from liquids increases, so does the demand for the ability to concentrate and/or remove particles from fluids.

### BRIEF DESCRIPTION OF THE DRAWING

FIG. 1 graphically illustrates a schematic view of an example microfluidic particle concentrator in accordance with examples of the present disclosure;

FIG. 2 graphically illustrates a schematic view of an example microfluidic particle concentrator in accordance with examples of the present disclosure;

FIG. 3 graphically illustrates a schematic view of an example microfluidic particle concentrator in accordance with examples of the present disclosure;

FIG. 4 graphically illustrates a schematic view of an example microfluidic particle concentrator in accordance with examples of the present disclosure;

FIG. 5 graphically illustrates a schematic view of an example microfluidic particle concentrator in accordance with examples of the present disclosure;

FIG. 6 graphically illustrates a schematic view of an example microfluidic particle concentrator in accordance with examples of the present disclosure;

FIG. 7 graphically illustrates a schematic view of an example microfluidic particle concentrator in accordance with examples of the present disclosure;

FIG. 8 graphically illustrates a schematic view of an example microfluidic particle concentrator in accordance with examples of the present disclosure;

FIG. 9 graphically illustrates an example particle concentrating system in accordance with examples of the present disclosure; and

FIG. 10 is a flow diagram illustrating an example method of concentrating particles in accordance with examples of the present disclosure.

### DETAILED DESCRIPTION

In many biological, chemical, and environmental assays, particles of interest can be present in very low concentrations. In accordance with examples of the present disclosure, by increasing the concentration of particles in a fixed liquid volume, detection of the particles otherwise at lower concentrations can occur, thus increasing the sensitivity of an assay. This can be an issue in circumstances were particulate concentrations may be highly diluted. For example, bacterial organisms can be present in liquids as rare as 1 organism for 100 mL of fluid. A large amount of fluid may be processed in order to obtain a small quantity of bacterial organisms. Thus, with some analysis protocols, testing may be difficult without concentrating the particles of interest, which may otherwise be present at a low concentration. By concentrating the particles from the sample fluid, analysis can occur (or can occur with greater resolution) in some examples. Alternatively, a fluid of interest may become more useful or may be more accurately evaluated after removal of particles therefrom, e.g., the portion that does not include the concentrated particles. In either or both instances, the particle concentration described herein can provide sample fluid for further use and/or assay of the sample fluid by transforming the initial sample fluid from a first state to multiple separate fluids with different particle concentrations.

In accordance with this, it is noted that the term “particles” refers to particulate materials of various types, including cells, microorganisms, undissolved analytes, other organic particulates, inorganic particulates, etc., that can be present in a sample fluid. In one example, the particles can be biological particles for biological assays or use, but other types of particles can likewise be concentrated. A “sample fluid” can refer to a fluid obtained for analysis and can include the particles to be concentrated or separate. The terms “particle-ablated” or “particle-concentrated” when referring to a sample fluid refers to the multiple portions of the sample fluid that remain after a plurality of particles are concentrated in accordance with the present disclosure. For example, during concentration of the particles, the portion that includes an increased concentration of particles can be referred to as the “particle-concentrated fluid” and the portion where particle concentration has been reduced can be referred to as “particle-ablated fluid.” Both are fluid portions that are generated from the source sample fluid. As a note, the source sample fluid can be of itself a previously “concentrated” or “ablated” sample fluid, as may be the case with cascading or sequential microfluidic particle concentrators.

In accordance with example of the present disclosure, a microfluidic particle concentrator includes an inlet microchannel, a filtering chamber fluidly connected to the inlet microchannel to receive a sample fluid, and a mechanical filter positioned in the filtering chamber. Additional features include a filter outlet microchannel fluidly connected to the filtering chamber to receive a particle-ablated fluid formed by passing through the mechanical filter, a particle outlet microchannel fluidly connected to the filtering chamber to receive a particle-concentrated fluid including a plurality of particles not permitted to pass through the mechanical filter, and a fluid movement network including multiple pumps. The pumps in this example generate sample fluid flow through the inlet microchannel and into the filtering chamber, particle-ablated fluid flow from the mechanical filter into the filter outlet microchannel, and/or particle-concentrated fluid from the filtering chamber into the particle outlet microchannel. In one example, the filtering chamber has an average cross-sectional size perpendicular to flow of the sample fluid ranging from 50 μm to 500 μm; and the inlet microchannel, the filter outlet microchannel, and the particle outlet microchannel individually has an average cross-sectional size perpendicular to flow of the sample fluid ranging from 1% to 40% of the cross-sectional size of the filtering chamber. In another example, the fluid movement network includes: an inlet pump within the inlet microchannel and a filter outlet pump within the filter outlet microchannel, an inlet pump within the inlet microchannel and a particle outlet pump within the particle outlet microchannel, a filter outlet pump within the filter outlet microchannel and a particle outlet pump within the particle outlet microchannel, or an inlet pump within the inlet microchannel, a filter outlet pump within the filter outlet microchannel, and a particle outlet pump within the particle outlet microchannel. In yet another example, the inlet pump includes an inertial pump, and one or both of the filter outlet pump or the particle outlet pump includes a fluid ejector. In a further example, the mechanical filter includes openings sized to disallow large particles having an average size from 5 μm to 50 μm to pass therethrough, and the particle outlet microchannel has an average cross-sectional size perpendicular to flow of the sample fluid ranging from 5% larger to 120% larger than a size of the largest particle of the large particles disallowed by the mechanical filter. In one other example, the mechanical filter includes a sieve, a baleen, a lateral displacement bar, a size exclusion chromatographic structure, or a combination thereof. In another example, the mechanical filter is tangentially oriented at an angle from 5° to 170° with respect to a direction of fluid flow through the filtering chamber and into the filter outlet microchannel, thereby directing larger particles disallowed by the mechanical filter toward the particle outlet microchannel. In yet another example, the microfluidic particle concentrator further includes an auxiliary filtering chamber fluidly connected to the filter outlet microchannel, wherein the auxiliary chamber includes an auxiliary mechanical filter, an auxiliary filter outlet microchannel, an auxiliary particle outlet, and an auxiliary fluid movement network. In a further example, the microfluidic particle concentrator further includes a coulter counter electrode operable to detect electrical resistance as the sample fluid passes therethrough. In another example, the particle outlet microchannel includes an auxiliary fluidic inlet to introduce an additional fluid into the particle outlet microchannel to separate droplets including particles from one another. In yet another example, the microfluidic particle concentrator further includes an auxiliary mechanical filter and an auxiliary particle outlet microchannel. The auxiliary mechanical filter is positioned in the filtering chamber prior to the mechanical filter along a fluid flow path, such that a sample fluid flowing through the microfluidic particle concentrator contacts the auxiliary mechanical filter prior to contacting the mechanical filter. The auxiliary mechanical filter directs a first stage of particle-concentrated fluid to the auxiliary particle outlet microchannel, while permitting a first stage of particle-ablated fluid to pass therethrough to be further separated at the by the mechanical filter to thereby form a second stage of particle-concentrated fluid and a second stage of particle-ablated fluid.

Also presented herein is a particle concentrating system. The particle concentrating system includes a microfluidic particle concentrator and a sample fluid. The microfluidic particle concentrator includes an inlet microchannel, a filtering chamber fluidly connected to the inlet microchannel to receive a sample fluid, a mechanical filter positioned in the filtering chamber, a filter outlet microchannel fluidly connected to the filtering chamber to receive a particle-ablated fluid formed by passing through the mechanical filter, a particle outlet microchannel fluidly connected to the filtering chamber to receive a particle-concentrated fluid including a plurality of particles not permitted to pass through the mechanical filter, and a fluid movement network including multiple pumps to generate sample fluid flow into the filtering chamber through the inlet microchannel, sample fluid flow out of the filtering chamber and into the filter outlet microchannel in the form of the particle-ablated fluid, and sample fluid flow out of the filtering chamber and into the particle outlet microchannel in the form of particle-concentrated fluid. The sample fluid including particles that are large enough for exclusion by the mechanical filter for concentration into the particle outlet microchannel. In one example, the particles are large enough for concentration and have an average particle size from 5 μm to 50 μm, and the mechanical filter is tangentially oriented at from 5° to 170° relative to direction of flow of the sample fluid through the filtering chamber to direct the particles large enough for concentration into the particle outlet microchannel.

In a further example, a method of concentrating particles is presented. The method includes, flowing a sample fluid including particles for concentration through an inlet microchannel and into a filtering chamber; filtering a first portion of the sample fluid to generate a particle ablated-fluid; flowing the particle-ablated fluid through a filter outlet microchannel; flowing a second portion of the sample fluid in the form of a particle-concentrated fluid through a particle outlet microchannel. As used herein, “particle ablated-fluid” refers to a fluid that has had the particles, or more typically, a portion of particles that were originally present removed by mechanical filtration. In one example, the flowing of the sample, flowing of the particle-ablated fluid, and flowing of the particle-concentrated fluid includes pumping with multiple pumps, including: an inlet pump within the inlet microchannel and a filter outlet pump within the filter outlet microchannel, an inlet pump within the inlet microchannel and a particle outlet pump within the particle outlet microchannel, a filter outlet pump within the filter outlet microchannel and a particle outlet pump within the particle outlet microchannel, or an inlet pump within the inlet microchannel, a filter outlet pump within the filter outlet microchannel, and a particle outlet pump within the particle outlet microchannel.

It is noted that when discussing the microfluidic particle concentrator, the particle concentrating system, or the method of concentrating particles herein, such discussions can be considered applicable to one another whether or not they are explicitly discussed in the context of that example. Thus, for example, when discussing an inlet microchannel in the context of a microfluidic particle concentrator, such disclosure is also relevant to and directly supported in the context of the particle concentrating system and/or the method of concentrating particles, and vice versa.

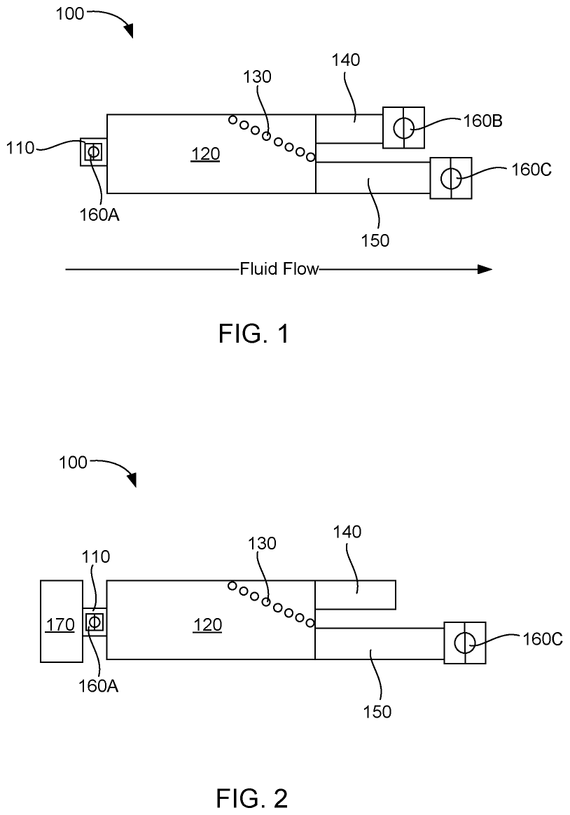

In accordance with these definitions and examples herein, FIGS. 1-8 depict various microfluidic particle concentrators at 100. These various examples can include various features, with some features common from example to example. Thus, the reference numerals used for FIGS. 1-8 are the same throughout to avoid redundancy, but it is understood that various other structural configurations can be used in accordance with the principles described herein. In FIGS. 1-8, with initial emphasis on the example shown in FIG. 1, the microfluidic particle concentrators can include an inlet microchannel 110, a filtering chamber 120, a mechanical filter 130, a filter outlet microchannel 140, a particle outlet microchannel 150, and a fluid movement network 160A, 160B, and/or 160C. The microfluidic particle concentrator can be used to concentrate particles having an average particle size ranging from 100 nm to 30 μm, from 500 nm to 20 μm, or from 750 nm to 15 μm. “Particle size” refers to the diameter of spherical particles, or to the longest dimension of non-spherical particles. Particle size can be measured by differential light scattering (DLS) or particle sizing via microscopic observation.

In further detail, an inlet microchannel can be structurally configured for depositing and receiving a sample fluid. In one example, an inlet microchannel can include an opening and a microchannel. The opening can provide fluid access. In a further example, the opening can be configured to include a fitting for connecting to a liquid dispenser, such as a syringe or a gas-tight syringe, or can include a fitting that can be penetrable by a liquid dispenser, such as a needle. The fitting for example, could include a male luer, female luer, threaded connector, bushing, elastomeric seal, or a tapered insert.

The microchannel of the inlet microchannel can be a chamber suitable for movement of fluid therethrough and can be fluidly connected to the filtering chamber. In one example, the microchannel of the inlet microchannel can have an average cross-sectional size perpendicular to flow of the sample fluid ranging from 1% to 40% of the cross-sectional size of the filtering chamber. In other examples, the microchannel of the inlet microchannel can have an average cross-sectional size perpendicular to flow of the sample fluid ranging from 5% to 25%, from 1% to 30%, or from 15% to 40% of the cross-sectional size of the filtering chamber.

The filtering chamber can be a linear chamber suitable for movement of a fluid therethrough. In one example, the filtering chamber can have an average cross-sectional size perpendicular to flow of the sample fluid ranging from 50 μm to 500 μm. In other examples, the filtering chamber can have an average cross-sectional size perpendicular to flow of the sample fluid ranging from 100 μm to 300 μm, from 75 μm to 250 μm, from 50 μm to 400 μm, or from 200 μm to 400 μm. An “average cross-sectional size” as used herein refers to a defined diameter if not circular, the diameter area of the cross-section reconfigured as a circular cross-section.

The filtering chamber can include a mechanical filter. The mechanical filter can include a sieve, baleen, lateral displacement bar, a size exclusion chromatographic structure, or a combination thereof. In one example, the mechanical filter can include multiple lateral displacement bars. When present, lateral displacement bars can include a space therebetween that can range from 10% to 200% of the particle size. In yet other examples of mechanical filters, the space therebetween can range from 10% to 20%, from 50% to 70%, from 110% to 200%, or from 90% to 110% of the particle size. In a further example, the mechanical filter can include a sieve.

In an example, the mechanical filter can include openings sized to prevent particles of interest from passing therethough. In one examples, the openings can be sized to prevent particles having an average size from 5 μm to 50 μm, from 5 μm to 17 μm, from 20 μm to 45 μm, from 15 μm to 35 μm, from 5 μm to 7 μm, from 9 μm to 12 μm, or from 12 μm to 17 μm passing therethrough. In yet other examples, the mechanical filter can include openings that can be larger than the particles of interest but can be positioned in a manner that minimizes the quantity of particles that pass therethrough.

In one example, the mechanical filter can be tangentially oriented at an angle from 5° to 170° with respect to a direction of fluid flow through the filtering chamber and into the filter outlet microchannel, thereby directing larger particles disallowed by the mechanical filter toward the particle outlet microchannel. In yet other examples, the mechanical filter can be tangentially oriented at an angle from 5° to 45°, from 30° to 150°, from 10° to 130°, or from 50° to 150° with respect to a direction of fluid flow through the filtering chamber and into the filter outlet microchannel, thereby directing larger particles disallowed by the mechanical filter toward the particle outlet microchannel. The angle and placement of the mechanical filter in the filtering chamber can direct particles that do not pass through the mechanical filter to the particle outlet microchannel.

In some example, the mechanical filter can be a tangential filter. Tangential filtration can be crossflow filtration where fluid flow occurs at an angle other than 90° in relation to the membrane face. In tangential filtration a relationship between mechanical filter and a direction of fluid flow can be at an angle other than 0° and 90° with respect to the relationship between one another.

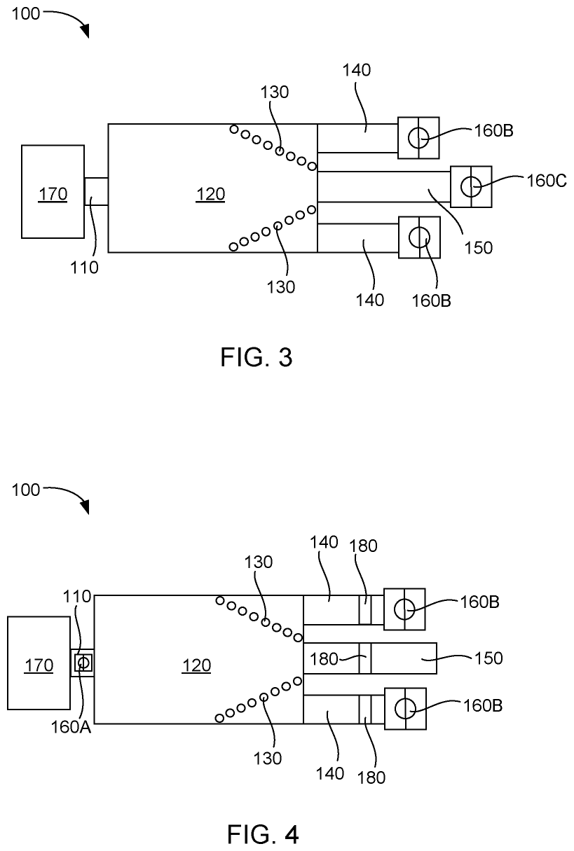

After passing through the mechanical filter, fluid with minimal quantities of particles of interest to fluid excluding the particles of interest, i.e. particle-ablated fluid can pass to the filter outlet microchannel. The filter outlet microchannel can be fluidly connected to the filtering chamber to receive a particle-ablated fluid formed by passing through the mechanical filter. In some examples, the microfluidic particle concentrator can include multiple mechanical filters and/or multiple filter outlet microchannels. An example microfluidic particle concentrators with multiple mechanical filters 130 and multiple filter outlet microchannels 140 is illustrated in FIG. 3 in a simple arrangement, as well as in FIGS. 4-8 with additional components, auxiliary mechanical filters 132 and other auxiliary components, etc.

In one example, a particle outlet microchannel can have an average cross-sectional size perpendicular to a flow of the sample fluid that can range from 5% larger to 120% larger than a size of the largest particle of the large particles disallowed by the mechanical filter. In yet other examples, the particle outlet microchannel can have an average cross-sectional size perpendicular to a flow of the sample fluid that can range from 15% larger to 100% larger, from 25% larger to 75% larger, or from 5% to 80% larger than a size of the largest particle of the large particles disallowed by the mechanical filter.

Particles that can be ablated from the fluid can be directed by the mechanical filter toward the particle outlet microchannel. The particle outlet microchannel can be fluidly connected to the filtering chamber to receive a particle-concentrated fluid including a plurality of particles that cannot be permitted to pass through the mechanical filter. The particle outlet microchannel can be fluidly connected to the filtering chamber. In some examples, the mechanical filter cannot extend over or across an opening to the particle outlet microchannel. In some examples, the particle outlet microchannel can have an average cross-sectional size perpendicular to flow of the sample fluid ranging from the 1% larger to 50% larger than a size of the largest particle of the large particles disallowed by the mechanical filter. In yet other examples, the particle outlet microchannel can have an average cross-sectional size perpendicular to flow of the sample fluid ranging from 5% larger to 35% larger, from 15% larger to 45% larger, or from 1% to 20% larger than a size of the largest particle of the particles disallowed by the mechanical filter.

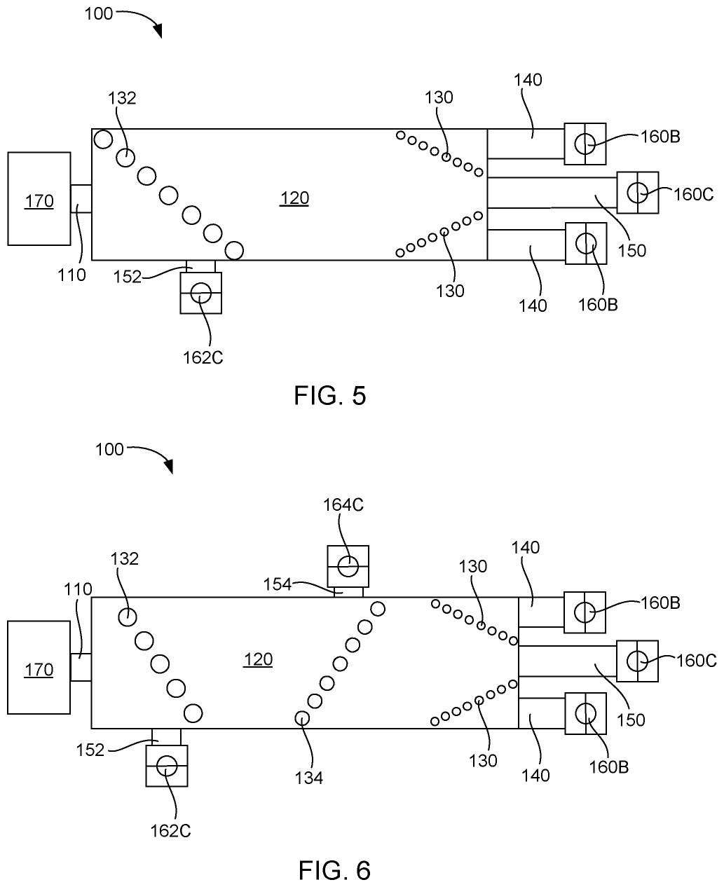

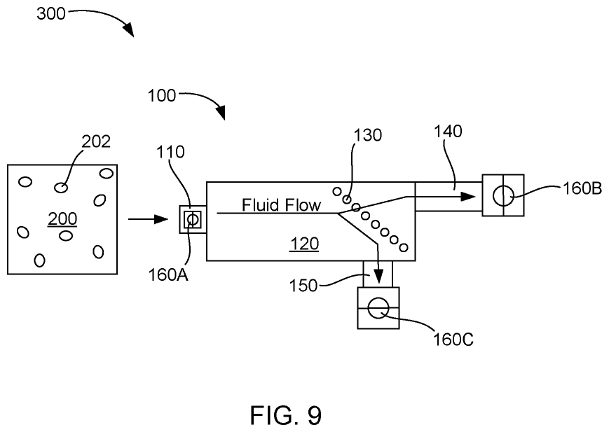

The location of the particle outlet microchannel can be parallel to fluid flow or can be perpendicular to fluid flow. For example, the particle outlet microchannel 150 can be located at the end of the filtering chamber as shown in FIGS. 1-8. In yet other examples, the particle outlet microchannel can be perpendicular to the filtering chamber as illustrated in FIG. 9, and/or as shown with respect to auxiliary particle outlet microchannels 152 and 154 illustrated in FIGS. 5 and 6. With more specific reference to FIG. 9, in addition to the perpendicular particle outlet microchannel (relative to fluid flows), similar to FIGS. 1-8, the microfluidic particle concentrator 100 can include an inlet microchannel 110, a filtering chamber 120, a mechanical filter 130, a filter outlet microchannel 140, the (perpendicular) particle outlet microchannel 150, and a fluid movement network 160A, 160B, and/or 160C.

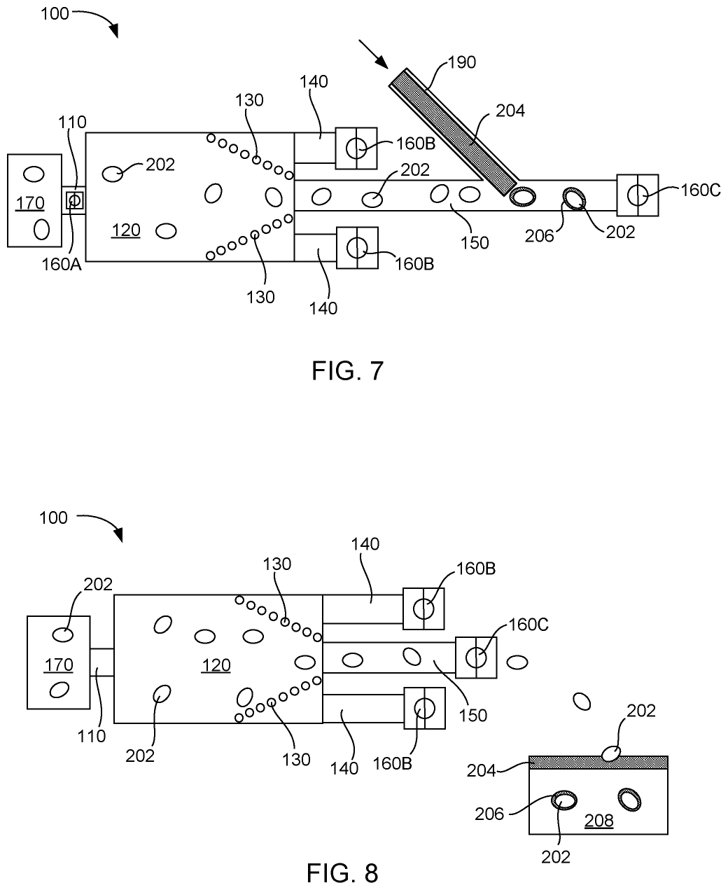

Regardless of the configuration shown in FIGS. 1-9, fluid flow through the microfluidic particle concentrator can be controlled by the fluid movement network 160A, 160B, and/or 160C (or others). The fluid movement network can include multiple pumps to generate sample fluid flow through the inlet microchannel and into the filtering chamber, particle-ablated fluid flow from the mechanical filter into the filter outlet microchannel, and particle-concentrated fluid from the filtering chamber into the particle outlet microchannel. The fluid movement network, for example, can include any combination of pumps that can generate fluid flow through the microfluidic particle concentrator. For example, the microfluidic particle concentrator can include an inlet pump 160A located within the inlet channel 110, a filter outlet pump 160B located in the filter outlet microchannel 140, and a particle outlet pump 160C located in the particle outlet microchannel 150 as illustrated in FIGS. 1 and 7. In another example, the microfluidic particle concentrator can include an inlet pump located in the inlet channel and a particle outlet pump located in the particle outlet microchannel as illustrated in FIG. 2. In a further example, the microfluidic particle concentrator can include a filter outlet pump located in the filter outlet microchannel and a particle outlet pump located in the particle outlet microchannel as illustrated in FIGS. 3, 5, 6, and 8. In yet another example, the microfluidic particle concentrator can include an inlet pump located in the inlet channel and a filter outlet pump located in the filter outlet microchannel, as illustrated in FIG. 4. Essentially, the location of the pumps can be at locations that drive fluid flow in the “Fluid Flow” direction shown in FIG. 1, and which causes particle concentration/separation to occur.

As shown by example in FIGS. 1-9, the various pumps 160A, 160B, and/or 160C can include an inertial pump, fluid or drop ejector, DC electroosmotic pump, AC electroosmotic pump, diaphragm pump, peristaltic pump, capillary pump, or a combination thereof. An inertial pump may in and of itself include multiple pumps that work together to generate a net unidirectional fluid flow. A fluid or drop ejector can include pumps that operate in the same way as piezo inkjet printheads or thermal inkjet printheads, ejecting fluid from one microfluidic channel in a direction away from the channel (and into a chamber, into another microfluidic channel, or to the environment outside of the microfluidic particle concentrator. In the examples shown, the pump at the inlet pump 160A located within the inlet channel 110 can generate fluid flow by “pushing” fluid through the inlet channel and into the filtering chamber 12. On the other hand, fluid ejectors 160B and/or 160B can generate a “pull” of fluid in the direction of the fluid flow. In one example, the inlet pump can include an inertial pump and one or both of the filter outlet pump or the particle outlet pump can include a fluid ejector. In another example, there can be ejectors at the filter outlet microchannel and the particle outlet microchannel. In another example, the pumps can be at both the inlet microchannel(s) and the various types of microchannel(s), for example. The combination of pumps can generate fluid flow through the microfluidic particle concentrator at a flow rate that can range from 10 pL/min to 50 mL/min. In other more specific example, the flow rate of fluid through the microfluidic particle concentrator can range from 10 pL/min to 30 mL/min, from 100 pL/min to 50 mL/min, from 1 mL/min to 50 mL/min, from 1 nL/min to 100 μL/min, from 10 10 nL/min to 100 nL/min, from 100 nL/min to 1 uL/min, or from 0.5 uL/min to 10 uL/min, for example. In some examples, the pump can include a thermal inkjet ejector, such as an ejector with 1,000 to 3,000 nozzles, e.g., about 2000 nozzles, pulling fluid therethrough at from 1 mL/min to 50 mL/min, e.g., about 30 mL/min.

In one example, the microfluidic particle concentrator can further include a fluid reservoir 170, as illustrated in FIGS. 2-8. A fluid reservoir can allow for loading of a volume of fluid larger than the volume that can pass through an inlet microchannel at a period of time. The reservoir can permit convenient fluid loading in some examples.

In another example, as shown by way of example in FIG. 4, the microfluidic particle concentrator can further include a coulter counter electrode 180, or multiple coulter counter electrodes, to detect electrical resistance as the sample fluid passes therethrough. A coulter counter electrode can be located at the filter outlet microchannel, the particle outlet microchannel, or a combination thereof. Detecting electrical resistance can permit the detection of individual particles, and/or a concentration of a solution as a fluid passes. A coulter counter electrode can provided added control to permit the ejection of specified quantities of particles. In some examples, a coulter counter electrode can be positioned at the filter outlet microchannel, the particle outlet microchannel, or the combination thereof.

In another example, as shown in FIG. 5 and FIG. 6, the microfluidic particle concentrator 100 can include additional mechanical filter(s) that are not specifically associated with a filter outlet microchannel 140, referred to herein as “auxiliary mechanical filter(s)” 132 and/or 134. The auxiliary mechanical filter can be as described above with respect to the mechanical filter, but may be positioned at other locations than those specifically associated with a filter outlet microchannel. For example, an auxiliary mechanical filter 132 may be associated with an auxiliary particle outlet microchannel 152 that may or may not include an auxiliary particle outlet microchannels 162C and/or 164C. These types of combinations can be used to remove larger particles before arriving at the mechanical filter 130, the filter outlet microchannel 140, and the particle outlet microchannel 150 described previously.

An auxiliary mechanical filter can filter particles of the same size or of a different size than particles that can be filtered by the mechanical filter. Filtering particles of the same size can minimize the potential for particles passing through the microfluidic particle concentrator uncollected. Filtering particles of a different size can permit separation and concentration of different sized particles in a single microfluidic particle concentrator.

An auxiliary mechanical filter can filter particles having a different size than particles filtered by a mechanical filter by varying the space between components of the auxiliary mechanical filter. For example, an auxiliary mechanical filter including lateral displacement bars can have a larger space between individual lateral displacement bars than a spacing between individual lateral displacement bars of a mechanical filter. In yet another example, an auxiliary mechanical filter including a sieve can have a larger spacing between the mesh than the spacing between the mesh of a mechanical filter including a sieve.

In some examples, auxiliary mechanical filters can be arranged in a plurality and the quantity of auxiliary mechanical filters is not limited. For example, the microfluidic particle concentrator can include two auxiliary mechanical filters as illustrated in FIG. 6. In yet other examples, the microfluidic particle concentrator can include a series of auxiliary mechanical filters. For example, a microfluidic particle concentrator can include from 3 to 20 auxiliary mechanical filters, from 3 to 8 auxiliary mechanical filters, or from 3 to 14 auxiliary mechanical filters.

An auxiliary mechanical filter can be positioned in the filtering chamber prior to the mechanical filter along a fluid flow path, such that a sample of fluid flowing through the microfluidic particle concentrator can contact the auxiliary mechanical filter prior to contacting the mechanical filter. The auxiliary mechanical filter can direct a first stage of particle-concentrated fluid to an auxiliary particle outlet microchannel, while permitting a first stage of particle-ablated fluid to pass therethrough to be further separated at the by the mechanical filter to thereby form a second stage of particle-concentrated fluid and a second stage of particle-ablated fluid. Auxiliary mechanical filters 132 and 134 associated with auxiliary particle outlet microchannels 162C and 164C are illustrated in FIG. 5 and FIG. 6.

In another example, as shown by way of example in FIG. 7, the microfluidic particle concentrator 100 can further include an auxiliary fluidic inlet 190 to introduce an additional fluid 204 into a particle outlet microchannel 150. For example, an additional fluid can be introduced as discrete droplets 206 that can separate portions of the fluid/particles found in the particle outlet microchannel from one another. The separation can be on a single particle-by-particle basis, or can include volumes of fluid with multiple particles therein. In this example, the additional fluid can provide a separation fluidic mechanism to accurately separately portions of concentrated particles for ejected through a particle outlet pump 160C, for example. An example of an additional fluid that can be introduced to separate droplets including particles and can include a solvent not miscible in the fluid that contains the particles. For example, the additional fluid can include stabilizers such as a surfactant. In one example the fluid can include mineral oil, AR20 polymethyl phenol siloxane, Isopar M, or a combination thereof.

In another example, as shown in FIG. 8, droplets 202 can be ejected from a particle outlet pump 160C, and the particles can be coated after ejection by passing through an auxiliary fluid 204 that is separated as a surface layer on second fluid 208 (hydrophobic layer/hydrophilic second fluid; or hydrophilic layer/hydrophobic second fluid) to be received within the second fluid as coated droplet suspended in the second fluid. Other architecture features can be similar to those described in FIGS. 1-7, for example, with any fluid movement network that generates fluid flow and particle separation as described herein.

In some examples, the microfluidic particle concentrator can include an auxiliary filtering chamber fluidly connected to the filter outlet microchannel. The auxiliary filtering chamber can include an auxiliary mechanical filter, an auxiliary filter outlet microchannel, an auxiliary particle outlet, and an auxiliary fluid movement network.

In one example, the microfluidic particle concentrator can be included as part of a microfluidic chip, such as a lab-on-a-chip device. The lab-on-a-chip device can be a point of care system. Incorporating the microfluidic particle concentrator in a lab-on-a-chip device can permit the analysis of reduced volumes of a sample fluid. For example, in biological assays including mammalian cells, bacterial cells, viruses, fungi, or the like, a particle of interest, such as a nucleic acid, protein, antibody, or the like, can be present in low concentrations. Thus, by increasing the concentration of the particle of interest, a reduced sample fluid volume can be used effectively in some instances. Applications can included concentrating particles for nucleic acid amplification, where concentrated nucleic acids may be moved into a microfluidic chamber(s) for amplification, e.g., an electrochemical cell, an optical detector, and/or thermal cycling cell to measure (electrical or optical) or initiate and carry out (thermal cycling) a polymerase chain reaction (PCR). Other assays and/or amplification processing that may occur using the concentrated particles may include strand displacement assays, transcription mediated assays, isothermal amplifications, loop mediated isothermal amplification, reverse-transcription loop mediated isothermal amplification, nucleic acid sequence based amplification, recombinase polymerase amplification, or multiple displacement amplification. In further examples, detection of concentrated particles, whether in the context of amplifying nucleic acids or some other concentrated particle application, can occur using electrical signal or optical signal detection equipment. Electrochemical signal can be measured, for example, using an electrochemical cell with a measuring electrodes, a counter-electrode, and a reference electrode, where an electrical signal (measured using the measuring electrode and the counter-electrode) may be detected and compared to a reference signal measured at the reference electrode. In other examples, concentrated particles can be detected optically, such as by using fluorescence, light scattering, or optical techniques.

In another example, as shown in FIG. 9, a particle concentrating system 300 can include a microfluidic particle concentrator 100 and a sample fluid 200. The microfluidic particle concentrator can include an inlet microchannel 110, a filtering chamber 120 fluidly connected to the inlet microchannel to receive a sample fluid, a mechanical filter 130 positioned in the filtering chamber, a filter outlet microchannel 140 fluidly connected to the filtering chamber to receive a particle-ablated fluid formed by passing through the mechanical filter, a particle outlet microchannel 150 fluidly connected to the filtering chamber to receive a particle-concentrated fluid including a plurality of particles not permitted to pass through the mechanical filter, and a fluid movement network 160A, 160B, and/or 160C, including multiple pumps (any combination of pumps at two or more of the following locations: inlet microchannel, filter outlet microchannel, or particle outlet microchannel, as described previously) to generate sample fluid flow in the direction shown in FIG. 9. Notably, as the particle outlet microchannel is oriented perpendicularly relative to the filter outlet microchannel, the fluid flow is considered to be into the general direction as shown, but in reality, a portion is directed perpendicularly to fluid flow through the filtering chamber. The microfluidic particle concentrator can be as described above. The sample fluid can include particles 202 that can be large enough for concentration into the particle outlet microchannel.

In one example, the particles large enough for concentration can have an average particle size ranging from 5 μm to 50 μm. In other examples, the particles large enough for concentration can have an average particle size that can range from 5 μm to 25 μm, from 10 μm to 20 μm, 7 μm to 12 μm, 5 μm to 50 μm, or from 25 μm to 35 μm. The mechanical filter can be tangentially oriented at from 5° to 170° relative to direction of flow of the sample fluid through the filtering chamber to direct the particles large enough for concentration into the particle outlet microchannel.

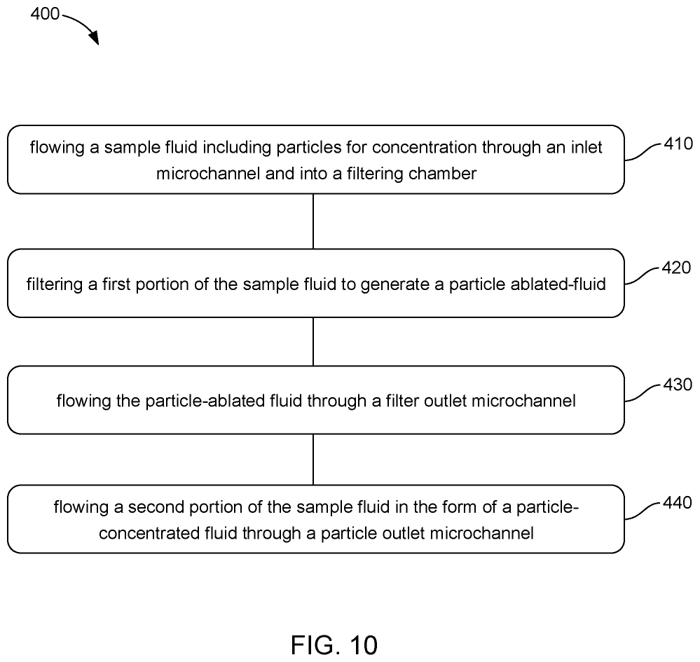

A flow diagram of a method 400 of concentrating particles is shown in FIG. 10. In one example, the method include flowing 410 a sample fluid including particles for concentration through an inlet microchannel and into a filtering chamber; filtering 420 a first portion of the sample fluid to generate a particle ablated-fluid; flowing 430 the particle-ablated fluid through a filter outlet microchannel; and 440 flowing a second portion of the sample fluid in the form of a particle-concentrated fluid through a particle outlet microchannel. The flowing of the sample, flowing the particle-ablated fluid, and flowing the particle-concentrated fluid can include pumping with multiple pumps. In one example, the multiple pumps can include an inlet pump within the inlet microchannel and a filter outlet pump within the filter outlet microchannel. In another example, the multiple pumps can include an inlet pump within the inlet microchannel and a particle outlet pump within the particle outlet microchannel. In another example, a filter outlet pump within the filter outlet microchannel and a particle outlet pump within the particle outlet microchannel. In a further example, an inlet pump within the inlet microchannel, a filter outlet pump within the filter outlet microchannel, and a particle outlet pump within the particle outlet microchannel.

It is noted that, as used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on presentation in a common group without indications to the contrary.

Concentrations, dimensions, amounts, and other numerical data may be presented herein in a range format. It is to be understood that such range format is used merely for convenience and brevity and should be interpreted flexibly to include the numerical values explicitly recited as the limits of the range, and also to include all the individual numerical values or subranges encompassed within that range as if each numerical value and sub-range is explicitly recited. For example, thickness from about 0.1 mm to about 0.5 mm should be interpreted to include the explicitly recited limits of 0.1 mm to 0.5 mm, and to include thicknesses such as about 0.1 mm and about 0.5 mm, as well as subranges such as about 0.2 mm to about 0.4 mm, about 0.2 mm to about 0.5 mm, about 0.1 mm to about 0.4 mm etc.

The terms, descriptions, and figures used herein are set forth by way of illustration and are not meant as limitations. Many variations are possible within the disclosure, which is intended to be defined by the following claims—and equivalents—in which all terms are meant in the broadest reasonable sense unless otherwise indicated.

What is claimed is:

1. A microfluidic particle concentrator, comprising
an inlet microchannel;
a filtering chamber fluidly connected to the inlet microchannel to receive a sample fluid;
a mechanical filter positioned in the filtering chamber;
a filter outlet microchannel fluidly connected to the filtering chamber to receive a particle-ablated fluid formed by passing through the mechanical filter;
a particle outlet microchannel fluidly connected to the filtering chamber to receive a particle-concentrated fluid including a plurality of particles not permitted to pass through the mechanical filter; and
a fluid movement network including multiple pumps to generate sample fluid flow through the inlet microchannel and into the filtering chamber, particle-ablated fluid flow from the mechanical filter into the filter outlet microchannel, and particle-concentrated fluid from the filtering chamber into the particle outlet microchannel.

2. The microfluidic particle concentrator of claim 1, wherein the filtering chamber has an average cross-sectional size perpendicular to flow of the sample fluid ranging from 50 μm to 500 μm; and wherein the inlet microchannel, the filter outlet microchannel, and the particle outlet microchannel individually have an average cross-sectional size perpendicular to flow of the sample fluid ranging from 1% to 40% of the cross-sectional size of the filtering chamber.

3. The microfluidic particle concentrator of claim 1, wherein the fluid movement network includes:
an inlet pump within the inlet microchannel and a filter outlet pump within the filter outlet microchannel,
an inlet pump within the inlet microchannel and a particle outlet pump within the particle outlet microchannel,
a filter outlet pump within the filter outlet microchannel and a particle outlet pump within the particle outlet microchannel, or
an inlet pump within the inlet microchannel, a filter outlet pump within the filter outlet microchannel, and a particle outlet pump within the particle outlet microchannel.

4. The microfluidic particle concentrator of claim 3, wherein the inlet pump includes an inertial pump, and one or both of the filter outlet pump or the particle outlet pump includes a fluid ejector.

5. The microfluidic particle concentrator of claim 1, wherein the mechanical filter includes openings sized to disallow large particles having an average size from 5 μm to 50 μm to pass therethrough, and wherein the particle outlet microchannel has an average cross-sectional size perpendicular to flow of the sample fluid ranging from 5% larger to 120% larger than a size of the largest particle of the large particles disallowed by the mechanical filter.

6. The microfluidic particle concentrator of claim 1, wherein the mechanical filter comprises a sieve, a baleen, a lateral displacement bar, a size exclusion chromatographic structure, or a combination thereof.

7. The microfluidic particle concentrator of claim 1, wherein the mechanical filter is tangentially oriented at an angle from 5° to 170° with respect to a direction of fluid flow through the filtering chamber and into the filter outlet microchannel, thereby directing larger particles disallowed by the mechanical filter toward the particle outlet microchannel.

8. The microfluidic particle concentrator of claim 1, further comprising an auxiliary filtering chamber fluidly connected to the filter outlet microchannel, wherein the auxiliary chamber includes an auxiliary mechanical filter, an auxiliary filter outlet microchannel, an auxiliary particle outlet, and an auxiliary fluid movement network.

9. The microfluidic particle concentrator of claim 1, further comprising a coulter counter electrode operable to detect electrical resistance as the sample fluid passes therethrough.

10. The microfluidic particle concentrator of claim 1, wherein the particle outlet microchannel includes an auxiliary fluidic inlet to introduce an additional fluid into the particle outlet microchannel to separate droplets including particles from one another.

11. The microfluidic particle concentrator of claim 1, further comprising an auxiliary mechanical filter and an auxiliary particle outlet microchannel, wherein the auxiliary mechanical filter is positioned in the filtering chamber prior to the mechanical filter along a fluid flow path, such that a sample fluid flowing through the microfluidic particle concentrator contacts the auxiliary mechanical filter prior to contacting the mechanical filter, wherein the auxiliary mechanical filter directs a first stage of particle-concentrated fluid to the auxiliary particle outlet microchannel, while permitting a first stage of particle-ablated fluid to pass therethrough to be further separated at the by the mechanical filter to thereby form a second stage of particle-concentrated fluid and a second stage of particle-ablated fluid.

12. A particle concentrating system, comprising:
a microfluidic particle concentrator, including:
an inlet microchannel,
a filtering chamber fluidly connected to the inlet microchannel to receive a sample fluid,
a mechanical filter positioned in the filtering chamber,
a filter outlet microchannel fluidly connected to the filtering chamber to receive a particle-ablated fluid formed by passing through the mechanical filter,
a particle outlet microchannel fluidly connected to the filtering chamber to receive a particle-concentrated fluid including a plurality of particles not permitted to pass through the mechanical filter, and
a fluid movement network including multiple pumps to generate sample fluid flow into the filtering chamber through the inlet microchannel, sample fluid flow out of the filtering chamber and into the filter outlet microchannel in the form of the particle-ablated fluid, and sample fluid flow out of the filtering chamber and into the particle outlet microchannel in the form of particle-concentrated fluid; and
a sample fluid including particles that are large enough for exclusion by the mechanical filter for concentration into the particle outlet microchannel.

13. The particle concentrating system of claim 12, wherein the particles large enough for concentration have an average particle size from 5 μm to 50 μm, and the mechanical filter is tangentially oriented at from 5° to 170° relative to direction of flow of the sample fluid through the filtering chamber to direct the particles large enough for concentration into the particle outlet microchannel.

14. A method of concentrating particles, comprising:
flowing a sample fluid including particles for concentration through an inlet microchannel and into a filtering chamber;
filtering a first portion of the sample fluid to generate a particle ablated-fluid;
flowing the particle-ablated fluid through a filter outlet microchannel;
flowing a second portion of the sample fluid in the form of a particle-concentrated fluid through a particle outlet microchannel.

15. The method of claim 14, wherein flowing the sample, flowing the particle-ablated fluid, and flowing the particle-concentrated fluid includes pumping with multiple pumps, including:
an inlet pump within the inlet microchannel and a filter outlet pump within the filter outlet microchannel,
an inlet pump within the inlet microchannel and a particle outlet pump within the particle outlet microchannel,
a filter outlet pump within the filter outlet microchannel and a particle outlet pump within the particle outlet microchannel, or
an inlet pump within the inlet microchannel, a filter outlet pump within the filter outlet microchannel, and a particle outlet pump within the particle outlet microchannel.
